(19) United States
(12) Patent Application Publication
(10) Pub. No.: US 2005/0271608 A1
(43) Pub. Date: Dec. 8, 2005

(54) Skin whitening compositions based on hydroxyaryl alkyl ketones and their isosteric derivatives

(76) Inventor: Shyam K. Gupta, Scottsdale, AZ (US)

Correspondence Address: SHYAM K. GUPTA;BIODERM RESEARCH, 5221 E. WINDROSE DRIVE, SCOTTSDALE, AZ, 85254, US

(21) Appl. No.: 10/862,037

(22) Filed: Jun. 5, 2004

(51) Int. Cl. A61K 7/135

(52) U.S. Cl. 424/62

(57) ABSTRACT

I have discovered that certain hydroxyaryl or polyhydroxyaryl compounds that contain an alkyl carbon side chain with a hetero-atom group attached by a double bond at the first carbon atom of the alkyl side chain that is directly attached to the aromatic ring, as illustrated in FIG. 1, provide a surprising and unexpected skin whitening effect. The addition of metal ions, such as copper, zinc, selenium, or vanadium and certain antioxidant compositions additionally increases the skin whitening effect.

### BACKGROUND OF INVENTION

The cosmetic treatment of skin to produce a visible even-tone has been practiced since ancient times. The use of plant-derived extracts and salves to whiten or brighten dark colored skin has been very popular among Asian, African, and South American cultures. The even toning of age-related dark spots, skin pigmentation, freckles, and other skin pigmentation disorders with skin lightening products is gaining popularity among people of light-colored skin as well. Hydroquinone is one of the Ingredients of choice, mostly because of its status as an FDA approved OTC drug active ingredient for skin whitening compositions. Kojic acid and arbutin, which are chemically related to hydroquinone, are also commonly used.

Topical applications of ascorbic acid and its esters are also claimed to have skin-lightening property. Several botanical-based ingredients with claims such as “helps reduce the appearance of minor skin discoloration”, “helps brighten skin and even-out skin tone”, “helps reduce the appearance of dark spots, age-related spots, and freckles”, and modifications of the above claims utilizing ascorbic acid and its derivatives have been disclosed. The color of human skin is differentiated by the nature and quantity of natural pigment, melanin, present in the epidermal layers of skin. The formation of melanin from amino acid tyrosine involves several biogenetic steps mediated initially by enzyme tyrosinase. Tyrosinase is a copper-based monooxygenase enzyme that catalyzes the hydroxylation of monophenols (hydroxybenzenes) and the oxidation of ortho-diphenols to ortho-quinones. This enzyme, found in prokaryotes as well as in eukaryotes, is involved in the formation of pigments such as melanins and other polyphenolic compounds. The active-site of tyrosinase is known to contain two copper ions (CuA and CuB). Each of the two copper ions has been shown to be bound by three conserved histidine residues. The regions around these copper-binding ligands are well conserved. Moreover, the distance between these two copper ions is 26 Angstrom units [(van Amsterdam et al., Angewandte Chemie, 42: 62-64 (2003); Bubacco et al., J. Biol. Chem., 181-194 (2003)]. At least two proteins related to tyrosinase are known to exist in mammals, and include TRP-1, which is responsible for the conversion of 5,6-dihydroxyindole-2-carboxylic acid (DHICA) to indole-5,6-quinone-2-carboxylic acid (IQCA) or indole-5,6-quinone (IQ); and TRP-2, which is the melanogenic enzyme DOPAchrome tautomerase that catalyzes the conversion of DOPAchrome to DHICA. TRP-2 differs from tyrosinases and TRP-1 in that it binds two zinc ions instead of copper. Other proteins that belong to this family are plant polyphenol oxidases (PPO), which catalyze the oxidation of mono- and ortho-diphenols to ortho-diquinone. From this discussion it should be clearly evident that any successful inhibition of tyrosinase at its active-site level is to be accomplished only by the blocking of both copper and zinc actives-sites. This should be possible by the use of appropriate transition state analogs. Moreover, any changes in the environment of copper-copper linkage at the active-site of tyrosinase that can result in the distortion of 26 Angstrom distance between those two copper atoms can also cause a disruption in the enzymatic activity of tyrosinase. These aspects for tyrosinase inhibition have been completely ignored by prior art disclosures for skin whitening compositions. This shall become clearer in the later discussions of the present invention.

The first step of tyrosinase action is the most critical because the remainder of the reaction sequence can proceed spontaneously at physiological pH. Here, tyrosinase converts tyrosine to dihydroxyphenylalanine (DOPA) and then to dopaquinone. Subsequently, dopaquinone is converted to dopachrome, through auto-oxidation, and finally to dihydroxyindole (DHI) or dihydroxyindole-2-carboxylic acid (DHICA), which form eumelanin (brown-black pigment). The latter reaction occurs in the presence of dopachrome tautomerase and DHICA oxidase. In the presence of cysteine or glutathione, dopaquinone is converted to cysteinyl DOPA or glutathione DOPA. Subsequently, pheomelanin, a yellow-red pigment, is formed. This is further discussed in U.S. Pat. No. 5,679,511 (Kwon). Skin and hair pigmentation is determined by the level of melanin present in the epidermis and hair fiber, respectively. For example, three different types of melanin are present in the epidermis: DHI-melanin, DHICA-melanin and pheomelanin. The different types of melanin vary in color or shade. DHI-melanin is the darkest and is blackish in color. DHICA-melanin is brownish in color. Pheomelanin is the lightest and is reddish in color. Pheomelanin is produced by the entrapment of dopachrome by sulfur-containing species such as cysteine and glutathione.

The successful development of a skin whitening formulation can incorporate three plausible approaches to skin depigmentation based on the mechanism of melanin formation discussed above; (1) The inhibition of melanin biosynthesis, (2) Possible conversion of melanin and its colored precursors (dopachrome, DHICA, DHI, IQCA) into colorless entities by reducing agents, and (3) inhibition of melanocyte stimulating hormone (MSH).

The inhibition of melanin biosynthesis may be achieved by the following mechanisms: (1) The competitive replacement of tyrosinase substrates, tyrosine or I-dopa with other chemically related compositions, (2) The inhibition of the oxidation/hydroxylation of tyrosine to produce I-dopa, (3) The inhibition of the conversion of I-dopa into dopaquinone, (4) The inhibition of the conversion of dopaquinone into dopachrome, (5) The inhibition of the conversion of dopachrome into DHICA or DHI, (6) The inhibition of the conversion of DHICA or DHI into IQCA or IQ, (7) The irreversible inactivation, replacement, or change in the metal-to-metal oxidation state of copper and zinc ion active-sites of TRP-1 and TRP-2. These mechanisms lead to the blocking of dark colored Eumelanin. An additional process to reduce the formation of darker colored Eumelanin is to promote the formation of Pheomelanin. For example, in the presence of cysteine or glutathlone, dopaquinone can be converted to cysteinyl DOPA or glutathione DOPA, which lead to the formation of less dark colored pheomelanin, (8) Changes in the environment of copper-copper linkage at the active-site of tyrosinase that can result in the distortion of 26 Angstrom distance between those two copper atoms, and (9) inhibition of melanocyte stimulating hormone (MSH). It has been generally known to utilize a tyrosinase inhibitor or a tyrosine competitor (to block enzyme tyrosinase), or a reducing agent (to convert melanin and its pigmented precursors into colorless, or less colored biochemical entities) by the prior art skin whitening compositions. A comprehensive treatment that encompasses a combination of the above biochemical mechanisms has been unknown until the present unexpected and surprising invention.

The present invention discloses novel skin whitening compositions that are based on (1) Biomimetic two-hydrogen donating competitive tyrosinase substrates, (2) inactivation of copper and zinc active-sites of TRP-1 and TRP-2, and (3) Optional inclusion of sulfhydryl (—SH) donating species to further retard the formation of Eumelanin. These three steps of the present invention surprisingly and unexpectedly accomplish all of the desired functions for the inhibition of melanin biosynthesis. The present invention is thus unprecedented, as previous disclosures have mostly centered on just one aspect of skin whitening; i.e. by tyrosinase inhibition.

A number of compositions that may act as competitive tyrosinase substrates have been reported. Chamaecin (2-hydroxy-4-isopropylbenzaldehyde) was synthesized and tested for its tyrosinase inhibitory activity. It partially inhibits the oxidation of 3, 4-dihydroxyphenylalanine (L-DOPA) catalyzed by mushroom tyrosinase, as reported by Nihel et al [Bloorg. Med. Chem. Lett., 14:681-3 (2004)]. The effects of thlohydroxyl compounds such as dithiothreltol (DTT) and beta-mercaptoethanol (beta-ME) on Cu2+at the active site have been elucidated. Treatment with DTT and beta-ME on mushroom tyrosinase completely inactivated 3,4-dihydroxyphenylalanine oxidase activity. Reactivation study of inactivated enzyme by addition of Cu2+confirmed that DTT and beta-ME directly bound with Cu2+at the active site, as reported by Park et al. [J Protein Chem., 22: 613-23 (2003)]. Anisic acid (p-methoxybenzoic acid) was characterized as a tyrosinase inhibitor from anise-seed, a common food spice. It inhibited the oxidation of L-3,4-dihydroxyphenylalanine (L-DOPA) catalyzed by tyrosinase. Anisic acid also inhibited the hydroxylation of L-tyrosine catalyzed by tyrosinase, as reported by Kubo et al. [Z Naturforsch [C], 58: 713-8 (2003)]. A kinetic study of the inhibition of mushroom tyrosinase by 4-substituted benzaldehydes showed that these compounds behave as classical competitive inhibitors, inhibiting the oxidation of L-3,4-dihydroxyphenylalanine (L-DOPA) by mushroom tyrosinase. It also inhibited the oxidation of L-tyrosine by mushroom tyrosinase (o-monophenolase activity) in a competitive manner, as reported by Jimenez et al. [J Agric Food Chem., 49: 4060-3 (2001)]. 2-Hydroxy-4-methoxybenzaldehyde was characterized as the principal tyrosinase inhibitor from three East African medicinal plants, the root of Mondia whitel (Hook) Skeels (Ascleplaceae), the root of Rhus vulgaris Meikle (Anacardiaceae), and the bark of Sclerocarya caffra Sond (Anacardlaceae). It inhibited the oxidation of L-3,4-dihydroxyphenylalanine (L-DOPA) by mushroom tyrosinase, as reported by Kubo et al. [Planta Med., 65 :19-22 (1999)].

A great number of skin whitening compositions have become commercially available. Most of those preparations are tyrosinase inhibitors. For example, Gattefosse markets “Gatulin Whitening”, which is a mixture of Aspergillus Orizae and Licorice extracts. Gattefosse also markets “Synerlight”, which is a mixture of Sophora root extract, kiwi water, and ascorbic acid, and “Mulberry Extract”, for skin whitening applications. “Etioline”, which is a mixture of Mitracarpus scaber extract and bearberry (Arctostaphylus uva ursi) extract, is skin whitening tyrosinase inhibitor marketed by Sederma. “Melaslow” and “Melaclear” are two additional skin-whitening compositions marketed by Sederma, both of which are based on tyrosinase inhibitors. “Dermalight” is a tyrosinase inhibiting composition based on nasturtium petals marketed by Silab. “Clariskin”, also offered by Silab, is another tyrosinase inhibitor derived from wheat germ extract. “Tyrostat-09” and “Tyrostat-11” marketed by Dragoco are both based on tyrosinase inhibitors obtained from a Canadian plant, and further disclosed in U.S. Pat. No. 6,521,267 (Steck). Alpaflor offers “Glgawhite”, a skin whitening composition based on a mixture of several tyrosinase inhibiting botanical extracts including Malva sylvestris, Mentha piperita, Primula veris, Alchemilla vulgaris, Veronica officinalis, Melissa offlcinalis, and Achillea millefolium. A much smaller number of compositions are available that generally act by reducing mechanism. The examples include hydroquinone, arbutin, and ascorbic acid and its derivatives.

Hydroquinone. An important industrial chemical, HQ is also a ubiquitous chemical readily available in cosmetic and nonprescription forms for skin lightening. it is considered one of the most effective inhibitors of melanogenesis in vitro and in vivo. HQ causes reversible inhibition of cellular metabolism by affecting both DNA and RNA synthesis. The cytotoxic effects of HQ are not limited to melanocytes, although the dose required to inhibit cellular metabolism is much higher for nonmelanotic cells than for melanocytes. Thus, HQ can be considered a potent melanocyte cytotoxic agent with relatively high melanocyte-specific cytotoxicity. HQ is also a poor substrate of tyrosinase, thereby competing for tyrosine oxidation in active melanocytes. The 2% HQ is readily available over-the-counter in various cosmetic preparations. However, for better efficacy, it often is compounded into various mixtures for treatment of hyperpigmentation. The original Kligman formula involves compounding 5% HQ with 0.1% retinoic acid and 0.1% dexamethasone in a hydrophilic ointment base. Concentrations as high as 10% can be compounded extemporaneously for refractory cases. Evidence of improvement with HQ (monotherapy) usually is observed at 4-6 weeks, with improvement appearing to plateau at about 4 months.

Despite its remarkable overall safety, the physician ought to bear in mind the potential adverse effects. Contact dermatitis occurs in a small number of patients and responds promptly to topical steroids. An uncommon, yet important, adverse effect of HQ is exogenous ochronosis. This disorder is characterized by progressive darkening of the area to which the cream containing HQ is applied. Histologically, degeneration of collagen and elastic fibers occurs. This degeneration is followed by the appearance of characteristic ochronotic deposits consisting of crescent-shaped, ochre-colored fibers in the dermis.

Exogenous ochronosis generally has been observed in black patients and after use of high concentrations of HQ for many years. However, cases occurring after the use of 2% HQ also have been reported. A South African epidemic of exogenous ochronosis due to HQ has been reported. For this reason, it generally is agreed that the use of HQ should be discontinued if no improvement occurs within 4-6 months. HQ-induced ochronosis often responds to topical steroids and chemical peeling. Lastly, HQ has been reported to induce mutations in Salmonella and at the hart locus of Chinese hamster V79 cells. Because of its potential mutagenic properties, HQ currently is banned in Europe and Japan for use as a depigmenting agent.

As indicated above, tretinoin has been used to enhance the efficacy of HQ. in a large-scale, double-blind, placebo-controlled study, 0.05% tretinoin caused a decrease in melanin content at 6 months. Two known inhibitors of glutathione, cystamine and buthionine sulfoximine, also have been reported for their enhancement of the inhibitory effect of HQ on pigmentation. The authors of the study reported a synergistic decrease in hair pigmentation when a combination of HQ (2% or 4%) and buthionine sulfoximine (5%) was applied to the dorsal skin of mice.

Monobenzyl ether of Hydroquinone. Like HQ, monobenzyl ether of hydroquinone (MBEH) belongs to the phenol/catechol class of chemical agents. However, unlike HQ, MBEH almost always causes nearly irreversible depigmentation of skin. Traces of MBEH have been found in disinfectants, germicides, rubber-covered dish trays, adhesive tape, powdered rubber condoms, and rubber aprons. in dermatology, MBEH should be used only to eliminate residual areas of normally pigmented skin in patients with refractory and generalized vitiligo. it has been suggested that the mechanism of depigmentation of MBEH is because of the selective melanocytic destruction through free radical formation and competitive inhibition of tyrosinase enzyme system.

U.S. Pat. No. 4,526,179 refers to certain hydroquinone fatty esters that have good activity and are less irritating and more stable than hydroquinone. Japanese Patent Application No. 27909/86 refers to other hydroquinone derivatives that do not have the drawbacks of hydroquinone but that have relatively poor efficacy. U.S. Pat. No. 5,449,518 refers to 2,5-dihydoxyphenyl carboxylic acid derivatives as skin depigmentation agents. However, it should be noted that several hydroquinone derivatives are potent allergens. For example primrose (Primula obconica) contains hydroquinone derivative, Miconidin (2-methoxy-6-pentyl-1 ,4-dihydroxybenzene) and its oxidation product, Primin (2-methoxy-6-pentyl-1 ,4-benzoquinone), both of which are allergens [(Peng Nan et al., Annals of Botany,91: 329-333 (2003)]. From the same plant methyl 2,4-dihydroxy-5-methyl benzoate (30.41%), methyl 2,6-dihydroxy-4-methyl benzoate (29.27%), and hypnone (8.92%) were also obtained, all of which were non-allergenic [(Na P et al., Nat Prod Letters, 16(4):249-53 (2002)]. in another species of primrose (Primula ovalifolia), Peng Nan et al. [(Z. Naturforsch. 58, 57-61 (2003)] reported the occurrence of acetyl hydroquinone and methyl acetyl hydroquinone, both of which were not studied for possible skin whitening effects by these authors.

N-Acetyl-4-S-cysteaminylphenol: Like HQ and MBEH, N-acetyl-4-S-cysteaminylphenol (4-S-CAP) belongs to the class of phenol/catechols. The acetyl derivative of 4-S-CAP appears to be an excellent substrate of tyrosinase substrate; it forms a melanin-like pigment when exposed to tyrosinase. Like HQ, it also is considered to be cytotoxic. in a study of 12 patients with melasma who used 4% 4-S-CAP, Jimbow [(Arch Dermatology, 127(10):1528-34 (1991)] reported a 66% improvement after 4 weeks of use. Furthermore, the author reported it to be more stable and less irritating than HQ.

Azelalc acid: A naturally occurring, saturated dicarboxylic acid originally isolated from Pityrosporum ovale, azelaic acid is a rather weak competitive inhibitor of tyrosinase in vitro. in addition, azelaic acid has an antiproliferative and cytotoxic effect on melanocytes. The latter effect is because of a rather potent inhibition of thioredoxin reductase, an enzyme involved in mitochondrial oxidoreductase activation and DNA synthesis. Azelaic acid is prescribed topically as a 20% cream and has been combined with glycolic acid (15% and 20%), and its efficacy has been compared with HQ 4% in the treatment of facial hyperpigmentation in dark-skinned patients. it has been reported that the combination formula was as effective as HQ 4% cream, although with a slightly higher rate of local irritation.

Kojic acid (5-hydoxy-2-methyl-4-pyran-4-one): A fungal metabolic product, kojic acid inhibits the catecholase activity of tyrosinase, which is the rate-limiting, essential enzyme in the biosynthesis of the skin pigment melanin. Kojic acid also is consumed widely in the Japanese diet with the belief that it is of benefit to health. Indeed, it has been shown to significantly enhance neutrophil phagocytosis and lymphocyte proliferation stimulated by phytohemagglutinin. Melanocytes treated with kojic acid become nondendritic with decreased melanin content. Additionally, it scavenges reactive oxygen species that are released excessively from cells or generated in tissue or blood. Kojic acid is used in concentrations ranging from 1-4%. Although effective as a skin-lightening gel, it has been reported to have high-sensitizing potential and cause irritant contact dermatitis. in a study comparing glycolic acid/kojic acid combination with glycolic acid/HQ, no statistical difference in efficacy existed between kojic acid and HQ. However, the kojic acid preparation was reported to be more irritating.

4-Hydroxyanisole: Like HQ, 4-hydroxyanisole (4HA) is cytotoxic to melanocytes. its clinical efficacy in inhibiting melanogenesis has been reported when used as a combination of 4HA 2% cream and 0.01% retinolc acid. The authors reported minimal local skin irritation with this combination. 4HA 2% alone did not produce significant hypo pigmentation.

Arbutin (hydroquinone-beta-D-glucopyranoside): A glycosylated HQ found at high concentrations in certain plants that are capable of surviving extreme and sustained dehydration, arbutin has been shown to inhibit melanin synthesis by inhibition of tyrosinase activity. This appears to be because of the inhibition of melanosomal tyrosinase activity, rather than the suppression of the synthesis and expression of this enzyme. Because arbutin does not hydrolyze to liberate HQ, the latter agent is not responsible for the inhibitory effect of arbutin on melanogenesis. Although the effective topical concentration in treating disorders of hyperpigmentation has not been formally evaluated and published, several manufacturers are marketing arbutin as a depigmenting agent.

Paper Mulberry: This tyrosinase inhibitor was isolated from a plant herbal extract. The plant roots from which paper mulberry was isolated were collected in Korea. The authors compared the tyrosinase inhibition of paper mulberry to kojic acid and HQ. IC₅₀, the concentration causing 50% inhibition of the activity of tyrosinase, was reported to be 0.396% compared to 5.5% for HQ and 10.0% for kojic acid. The authors also performed a patch test using 1% paper mulberry extract and found no significant irritation at either 24 hours or 28 hours.

Glabridin: Glabridin is the main ingredient in licorice extract. The authors investigated glabridin for its inhibitory effect on pigmentation and reported that glabridin inhibited tyrosinase activity of melanocytes without any cytotoxicity. They further showed that UV-Binduced pigmentation and erythema was inhibited by topical application of 0.5% glabridin. The anti-inflammatory properties of glabridin were attributed to inhibition of superoxide anion production and cyclooxygenase activity.

Arctostaphylos patula and Arctostaphylos viscida: The leaves of these two Arctostaphylosplants have been reported to be potent inhibitors of tyrosinase. These two extracts not only inhibited the production of melanin from dopachrome but also exhibited superoxide dismutase-like activity. The effective topical concentration of these two plants in disorders of hyperpigmentation currently is not known.

Melatonin: Melatonin is secreted by the pineal gland in response to sunlight. This pineal agent is considered to be responsible for lightening the color of amphibians. When added to cultures of hair follicles of the Siberian hamster, melatonin was shown to bring about a dose-related inhibition of melanogenesis. However, tyrosinase activity was not affected, suggesting that the inhibition of melanogenesis occurs at the post-tyrosinase step in the melanin biosynthetic pathway. Melatonin has been shown to inhibit adenosine 3′,5′-cyclic phosphate (cAMP) driven processes in pigment cells. The concentration for topical use of melatonin for hyperpigmentatlon disorders has not been formally established. However, topical melatonin also has been reported to have anti-inflammatory properties when applied at 0.6 mg/cm². A cosmetic manufacturer currently producing and marketing topical melatonin cream reports melatonin as an effective antioxidant when topically applied at a concentration of 1%.

Magnesium ascorbyl phosphate: Magnesium ascorbyl-2-phosphate (MAP) is a stable derivative of ascorbic acid. When used as a 10% cream, MAP was shown to suppress melanin formation. A significant lightening effect was seen clinically in 19 of 34 patients with melasma and solar lentigos. Furthermore, MAP has been shown to have a protective effect against skin damage induced by UV-B irradiation. The latter protective effect is because of the conversion of MAP to AS.

The author of the present invention has published an article on skin whitening agents (S.Gupta, “Plant-based Skin Whitening Cosmetics”, Household and Personal Products industry (HAPPI), page 90, April 2001). Some of the important compositions include Paper Mulberry (Broussonetia kazinoke); the extracts of root and bark are potent tyrosinase inhibitors. Mitracarpe (Mitracarpus scaber), the leaf extract of this tropical plant contains harounoside, a hydroquinone analog with strong anti-tyrosinase activity. Also, a mixture of this extract with bearberry has shown potent tyrosinase inhibiting and skin whitening properties. Bearberry (Arctostaphylos uva ursi), the leaf extract of this plant contains hydroquinone derivatives, arbutin and methyl arbutin with skin whitening attributes. Yellow Dock (Rumex crispus, Rumex occidentalls), this extract has shown excellent anti-tyrosinase and skin whitening attributes. The chemical constituents of this recently discovered material responsible for skin whitening activity are unknown. Glutathione: Glutathione has been used in skin lightening compositions. Reduced form of glutathione has a dual role in the depigmentation of colored skin. The mechanism of action may involve competitive binding with the color forming precursors of tyrosine (dopaquinone) to form less-colored phaeomelanin. it may also act as a reducing agent to effect the bleaching of the colored melanin precursors. Leukocyte Extract: it is a fractionated blend of biotechnology-derived peptides with tyrosinase inhibiting activity. The possible mode of action may involve its ability to denature protein backbone of enzyme tyrosinase, thus inhibiting that enzyme. Aspergillus orizae: this fermentation-derived material contains kojic and lactic acids. Kojic acid is a known tyrosinase-inhibiting, skin color-reducing Ingredient. Licorice Root (Glycyrrhiza glabra): this botanical has been used for a variety of skin disorders since ancient history. Recent studies have shown its promising skin whitening activity. Hispaglabridin, glabridin, isoliquiritin, and their derivatives present in this botanical have striking structural similarity to other dihydroxybenzene-type skin whitening compounds. Rosmarinic Acid, Tetrahydrocurcumin, and Green Tea Extract: these all possess antityrosinase activity and skin lightening properties. Yohimbe (Pausinystalia yohimbe), the extract of yohimbe bark contains alkaloid Yohimbine and its isomers. it is reported to inhibit melanin biosynthesis, hence its application in cosmetic skin bleaching formulations. Cang Xu, Bal Xu: These Chinese folk medicines have been used for centuries for skin whitening, age spot removal, and skin tone enhancement applications. These are known to possess tyrosinase-inhibiting effects. The rhizomes of Cang Xu (Atractylodes lancea) and related plant, Bai Xu (Atractylodes macrocephalia) have been used for at least 2000 years in China for the removal of dark spots on the face and hand and skin whitening liniments. Atractylodin, an acetylenic furan derivative present in high amounts in these extracts may be a tyrosinase inhibitor. Bai Xian Pi (Dictamnus albus) preparations from root bark have antifungal and skin whitening attributes. Hu Zhang (Polygonum cuspidatum) has been used in China with a recorded history of over 2000 years. it contains anthraquinone derivative, emodin and stilbene derivative, resveratrol, which have recently been shown to possess tyrosinase-inhibiting activity. Gao Ben (Ligusticum sinensis) and its close relative, Chuanxiong (Ligusticum chuanxiong) have been used for dark spots, freckles, acne rosacea, and skin whitening applications dating back to 800 B.C. Ferulic acid present in these extracts may be a tyrosinase inhibitor. Fangfeng (Saposhnikovia divaricata) has been used for dark spots removal and skin whitening preparations. it has also been known that UV and sunlight stimulate the production of melanin as a result of body“s own protective response to such external stimuli. The use of sunscreen compositions has been practiced to reduce such skin darkening effects of UV and sunlight.

Relative to prior art knowledge of skin whitening compositions, several examples can be cited. To date, the best-known active substance for de-pigmentation is hydroquinone, a bleaching agent. Hydroquinone, however, does not inhibit melanin biosynthesis; it bleaches existing melanin. if applied over long periods of time, hydroquinone can have serious side effects, which has led to its being permitted only in limited concentrations in some countries, and to its being completely forbidden for applications in cosmetic products in other countries. Furthermore, hydroquinone leads to permanent de-pigmentation, and thus to increased photosensitivity of the skin when exposed to UV light. Better-tolerated skin lightening substances currently being used are of natural origin, e.g., arbutin (from the leaves of the common bearberry, Uva ursi), licorice extract (from liquorice root), ascorbic acid (vitamin C from citrus fruits) and their derivatives, as well as kojic acid (from carbohydrate solutions under the effect of certain bacteria). These substances, which are highly soluble in water, act on the tyrosinase as competitive inhibitors; however, they are unstable in some formulations, and have the disadvantage that only very small quantities penetrate the deeper skin layers and reach the melanocytes in the basal membrane. A further disadvantage of these substances is their low level of efficacy, which necessitates their being used in high concentrations. Compared to the quantity of hydroquinone used, 17 times as much ascorbic acid and over 100 times as much arbutin is required to achieve a similar effect.

U.S. Patent Application 20040047931 (Arquette) discloses compositions based on Simmondsin and Jojoba extract for skin whitening applications.

U.S. Patent Application 20040042984 (Park et al.) discloses depigmenting composition containing arbutin and glucosidase as active ingredients. The glucosidase is an enzyme hydrolyzing arbutin into hydroquinone and glucose. in the composition of this invention, arbutin and glucosidase are separated and mixed Just before applying to the skin. Then arbutin hydrolyzes into hydroquinone and glucose and the whitening effects are achieved by the hydroquinone inhibiting melanogenesis. This process is thus not very convenient, and the efficacy is significantly dependent on the activity of glucosidase enzyme.

Resorcinol derivatives have cosmetic skin and hair benefits. Certain resorcinol derivatives, particularly 4-substituted resorcinol derivatives, are useful in cosmetic compositions for skin lightening benefits. Resorcinol derivatives are described in many publications, including Hu et al., U.S. Pat. No. 6,132,740 and European Patent Application EP 1134 207; and Japanese published patent applications JP 2001-010925 and JP2000-327557. Resorcinol derivatives are known compounds and can be readily obtained by various means, including by a method wherein a saturated carboxylic acid and resorcinol are condensed in the presence of zinc chloride and the resultant condensate is reduced with zinc amalgam/hydrochloric acid (Lille, et al., Tr. Nauch-Issled. inst. Slantsev 1969, No. 18:127-134), or by a method wherein resorcinol and a corresponding alkyl alcohol are reacted in the presence of an alumina catalyst at a high temperature of from 200 to 400.degree. C. (British Patent No. 1,581,428). Some of these compounds can be irritating to the skin. U.S. Patent Application 20030190298 (Bradley) relates to certain resorcinol derivatives and their use as skin lightening agents. U.S. Patent Application 20040042983 (Harichian) discloses certain hydroxycoumarin derivatives, which are structurally similar to resorcinol derivatives, as skin whitening compositions.

U.S. Patent Application 20030129259 (Mahalingam et al.) disclose compositions for skin whitening by inhibiting enzymes other than tyrosinase, specifically DOPAchrome tautomerase (DCT) and/or 5,6-dihydroxyindole-2-carboxylic acid polymerase (DHICA-polymerase).

U.S. Patent Application 20040028712 (Shepherd) discloses skin brightening compositions that are characterized by the presence of leukocyte extract in combination with one or more secondary skin whitening or brightening agents. A preferred group of such agents includes bearberry, arbutin, rutin, ascorbyl glucoside, ascorbyl magnesium phosphate, hydroquinone, kojic acid or combinations thereof. Another group of preferred agents includes extracts of mulberry, lemon, orange, licorice, cucumber, cinnamon, cherry (fermentate), rosemary and/or derivatives thereof.

U.S. Patent Application 20040086560 (Chaudhuri et al.) discloses skin whitening effects of the extract of Emblica Officinalis.

U.S. Patent Application 20040092586 (Ogata et al.) discloses skin whitening benefits of alpha-lipoic acid.

U.S. Patent Application 20040018159 (Shinpu et al.) discloses chamomile extract as skin whitening agent.

U.S. Patent Application 20030190299 (Sakamoto et al.) discloses compositions based on ergosterol derivatives for skin whitening.

U.S. Patent Application 20030133958 (Kuno et al.) disclose skin whitening compositions comprising at least one member selected from the triterpenoid class of compounds consisting of maslinic acid, erythrodiol, uvaol, betulinic acid, and betulin.

U.S. Patent Application 20030152536 (Pauly et al.) discloses skin whitening compositions containing extracts of Waltheria indica and ascorbic acid, ferulic acid and/or kojic acid.

U.S. Patent Application 20030077236 (Mammone et al.) discloses a skin whitening composition based on Ascomycete-derived melanin-degrading enzyme extract.

U.S. Patent Application 20030049213 (Matsuda et al.) discloses skin whitening compositions based on melanin synthesis inhibiting macrocyclic derivatives such as cyclotetradecanone, cyclopentadecanone, cyclohexadecanone, cycloheptadecanone, cyclooctadecanone, cyclononadecanone, cycloelcosanone, cycloheneicosanone, cyclodocosanone, cyclotricosanone, cyclotetracosanone, cyclopentacosanone, 3-methylcyclopentadecanone, (S)-3-methylcyclopentadecanone, (R)-3-methylcyclopentadecanone, 3-methylcyclohexadecanone, 4-methylcyclohexadecanone, 4-cyclopentadecenone, 5-cyclopentadecenone, 4-cyclohexadecenone, 5-cyclohexadecenone, (E)-5-cyclohexadecenone, (Z)-5-cyclohexadecenone, 9-cyclopentadecenone, (E)-9-cyclopentadecenone, (Z)-9-cyclopentadecenone, 3-methyl-4-cyclopentadecenone, 3-methyl-5-cyclopentadecenone, 3-methyl-4-cyclohexadecenone, 3-methyl-5-cyclohexadecenone, 4-methyl-4-cyclohexadecenone, 4-methyl-5-cyclohexadecenone, 10-cycloelcosenone, 11-cyclodocosenone and 12-cyclotetracosenone. These compositions do not contain a hydroxyaryl or polyhydroxyaryl composition that contains an alkyl carbon side chain with a hetero-atom group attached by a double bond at the first carbon atom of the alkyl side chain that is directly attached to the aromatic ring.

U.S. Patent Application 20030017997 (Yokota et al.) discloses dihydroxyphenyl ketone derivatives derived from gingerone as skin whitening compositions. However, in gingerone the alkyl carbon directly attached to the aromatic ring does not contain a hetero-atom group attached by a double bond at the first carbon atom of the alkyl side chain. in fact, it is attached at the third carbon atom of the alkyl side chain of the dihydroxyphenyl ketone derivatives of Yokota et al. in fact, the dihydroxyphenyl alkyl ketone derivatives with such distantly bonded hetero-atom group attached by a double bond (ketone group in this case) are known to be stabilizers of color (and not whitening agents), as disclosed in U.S. Patent Application 20040091589 (Roy et al.).

U.S. Patent Application 20020176903 (Kuno et al.) discloses olive extracts for skin whitening benefits. These appear to be tyrosinase inhibitors.

U.S. Patent Application 20030103916 (Imanaka et al.) disclose skin-whitening cosmetic that comprises (A) at least one member selected from unsaturated fatty acids having from 18 to 22 carbon atoms and having from 2 to 6 double bonds in the molecular structure, and derivatives thereof, (B) a phospholipid, (C) an antioxidant, (D) at least one member selected from proteins and hydrolyzates thereof, and (E) at least one member selected from muco-polysaccharides and salts thereof.

U.S. Patent Application 20020141956 and 20020071816 (Perricone) disclose hydroxytetronic acid and its derivatives as anti-tyrosinase skin whitening compositions.

U.S. Patent Application 20010029253 (Park et al.) discloses certain 1,3-beta-glucan derivatives for skin whitening applications.

U.S. Patent No. 6,521,267 (Steck) discloses tyrosinase inhibiting extract derived from dicotyledonous plant species indigenous to Canada. U.S. Patent No. 6,365,137 (Aust et al.) discloses certain polyhydroxycarboxylic acids an anti-tyrosinase skin whitening agents.

U.S. Patent No. 6,083,976 (Padmapriya et al.) discloses certain aloesin derivatives as tyrosinase inhibiting skin whitening agents. U.S. Patent No. 5,993,835 (Mishima) discloses certain melanin complexing agents for skin whitening applications.

U.S. Patent No. 5,980,904 (Leverett et al.) discloses a skin whitening composition that contains bearberry extract and a reducing agent to boost the skin-whitening efficacy of bearberry extract. The reducing agents of preferred compositions were all formaldehyde donors. The formaldehyde donors are forbidden in many countries of the world due to their toxicity and skin sensitization problems.

U.S. Patent No. 5,91 6,915 (Hong et al.) discloses certain ascorbic acid derivatives for skin whitening applications.

U.S. Patent 5,824,327 (Whittemore et al.) discloses certain derivatives of kojic acid, suitable as tyrosinase inhibiting skin-whitening agents. These derivatives circumvent the problems of other kojic acid derivatives, for example Nagai, et al, U.S. Pat. No. 4,278,656 forms a skin whitener cosmetic composition from a kojic acid ester with an aliphatic carboxylic acid. The composition utilizes water and the non-dipalmitate esters used will turn color. The U.S. Pat. No. 4,369,174 to Nagai, et al, discloses a skin whitener cosmetic composition utilizing a kojic acid ester as an active Ingredient. The composition utilizes water and will turn color. in the U.S. Pat. No. 4,696,813 to Higa, the skin whitener cosmetic composition comprises placenta and kojic acid. it does not use esters, does utilize water and will turn color.

Hatae, et al, U.S. Pat. No. 4,847,074 relates to a kojic acid containing whitener cosmetic composition that includes cyclodextrins for improved stability, i.e. to compensate for color changes. U.S. Pat. No. 4,919,921 (Hatal) the cosmetic composition comprises kojic acid or an ester of kojic acid and Vitamin C, but this composition will change color in formulation. Hara, U.S. Pat. No. 4,948,577 relates to a skin whitener composition comprised of kojic acid or derivatives thereof with 4-(1,1-dimethylethyl)-4.sup.1 methoxydiben-zoylmethan formulated therein. The composition will turn color, and prevents normal tanning but is not true skin bleach. The skin whitener composition of U.S. Pat. No. 4,985,255 (Higa) comprises placental extract of pregnant cows and kojic acid or a derivative thereof. The mixture uses water and will turn color. U.S. Pat. No. 4,985,455 (Motono) incorporates an ultraviolet absorber, B cyclodextrin and ethylendiaminetetracetic acid to eliminate or reduce discoloration in a kojic acid or derivatives thereof skin whitener cosmetic composition.

U.S. Pat. No. 4,990,330 (Oyama) an amino compound is included in a kojic acid based skin whitener cosmetic composition to inhibit melanin synthesis. The skin lightening composition of Meybeck, et al, (U.S. Pat. No. 5,164,182) is a composition containing a mulberry extract incorporated into hydrated lipidic lamellar phases of liposomes. in the whitener composition of Maybeck, U.S. Pat. No. 5,279,834 hydroquinone and/or kojic acid or a derivative thereof is partially incorporated into liposomes.

U.S. Pat. No. 5,427,775 (Sakai) the whitening composition comprises teprenone and one or more substances selected from the groups consisting of kojic acid, L-ascorbic acid and arbutin. U.S. Pat. No. 5,609,875 (Hadas) discloses licorice extract compositions that possess boosted skin whitening efficacy from the inclusion of certain hydroxy acids. Since hydroxy acids themselves are known for their skin whitening property, such a disclosure is not surprising or unexpected.

U.S. Pat. No. 6,214,352 (Matsukawa) discloses extracts of Gardenia, Sophora and Rosa species of plants to possess tyrosinase-inhibiting activity. U.S. Pat. No. 6,165,982 (Yamada) discloses compositions with sericin to be tyrosinase inhibitors for skin whitening application.

U.S. Pat. No. 5,773,014 (Perrier et al.) discloses tyrosinase-inhibiting properties of extracts of mulberry, saxifrage, grape and scutellaria root. The above references again illustrate that tyrosinase inhibitors represent the greatest number of skin whitening compositions.

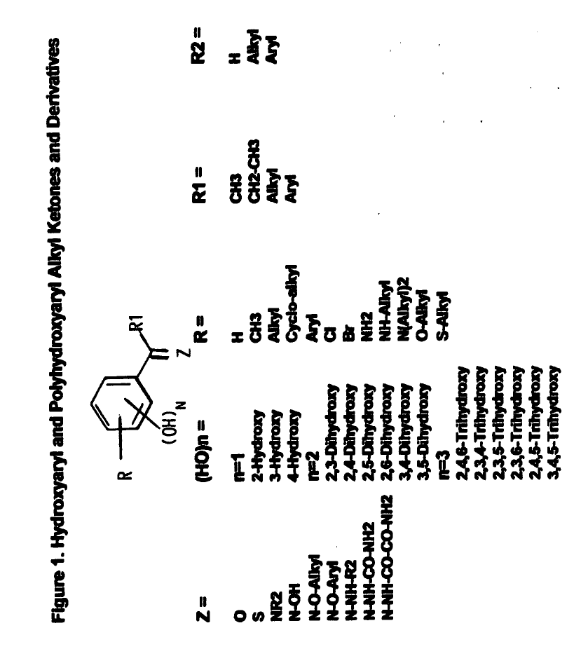

On the basis of foregoing discussion, the following key factors become evident that can control skin depigmentation: (1) Inhibition of melanin biosynthesis; and (2) Conversion of melanin or melanin precursors to decolorized forms; and (3) Inhibition of tyrosinase enzyme; and (4) Competitive replacement of tyrosinase substrates; and (5) Distortion of copper-copper bond of tyrosinase active-site (which is at 26 Angstrom units), and (6) Inhibition of MSH. it is clear that a complete solution to this problem has not been achieved by any of the prior art disclosures. Surprisingly, it has now been discovered that certain hydroxyaryl derivatives that also contain a carbon side chain directly attached to the aromatic ring carbon, and this carbon chain also contains a hetero-atom group at the first carbon atom of the side chain that is directly attached to the aromatic ring carbon position (see FIG. 1), provide skin whitening benefits.

[FIG. 1. Hydroxyaryl and Polyhydroxyaryl Alkyl Ketones and Derivatives]

The skin whitening effect is further increased if the hydroxyl group of the hydroxyaryl derivative is on a carbon atom adjacent to the carbon atom that also contains aforementioned carbon side chain with a hetero-atom group attached by a double bond at the first carbon atom of the side chain that is directly attached to the aromatic ring carbon position. The skin whitening effect is frequently enhanced further if additional hydroxy groups are also present on the aromatic ring. These chemical structural criteria are further illustrated in FIG. 1. Moreover, the skin whitening effect Is further enhanced if small amounts of divalent and polyvalent metal ions (such as copper, zinc, manganese, selenium, or vanadium) are also present.

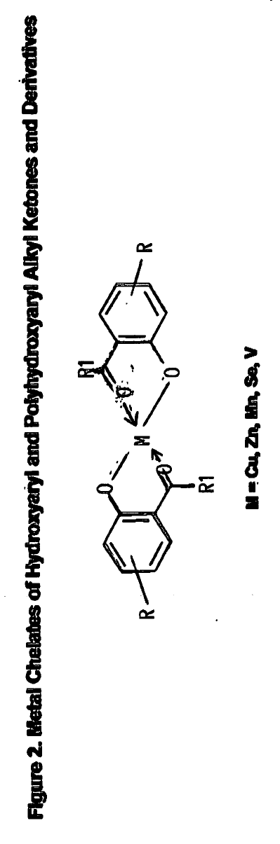

Although not bound by any specific scientific data, it is theorized that the skin whitening effect of hydroxyaryl derivatives is due to the following mechanisms, (1) Competitive replacement of tyrosinase substrates, (2) The irreversible inactivation, replacement, or change in the metal-to-metal oxidation state of copper and zinc ion active-sites of TRP-1 and TRP-2. These mechanisms lead to the blocking of dark colored Eumelanin., (3) Inhibition of MSH, and (4) Conversion of melanin or melanin precursors to decolorized forms. The enhancement of skin whitening effect of hydroxyaryl compounds of the present invention by trace levels of certain divalent and polyvalent metal ions (such as copper and zinc ions) is possibly due to the formation of corresponding metal chelate derivatives of such hydroxyaryl compounds (FIG. 2). Such chelates can cause distortion of 26 Angstrom units copper-copper bond of tyrosinase active-site. Thus, several biochemical mechanisms are performed by the compositions of present invention simultaneously to effect skin whitening, which is unprecedented in prior art.

Definitions: For the purpose of clarification, various definitions are described below.

Chelating: A chemical group present on an organic molecule that can form a chemical bond, called a chelate bond or chelate complex, with a metal atom (usually a divalent or polyvalent metal ion).

Chelating Hydroxy Aryl : Any aromatic compound that also has at least one hydroxyl group and at least one another group (henceforth called a “chelating”group), which in combination with hydroxyl group, can form a chelating chemical bond with a divalent or trivalent metal atom. Moreover, both hydroxyl group and chelating group are present on the aromatic ring adjacent to each other.

Hydroxy Aryl: Any of aromatic hydrocarbons and aromatic heterocyclic compounds that also contain a hydroxyl group directly attached to the aromatic hydrocarbon or aromatic heterocyclic ring.

Skin Whitening or Skin Lightening: Any of a number of compositions that can cause the lightening of skin color of people with lighter skin color (such as Caucasian or Oriental skin types), and whitening of skin color of people with darker skin color (such as African, Hispanic, and Indian Subcontinent skin types). These phrases are frequently used interchangeably.

### SUMMARY OF INVENTION

I have now discovered that certain hydroxyaryl derivatives that also contain a carbon side chain directly attached to the aromatic ring carbon, and this carbon chain also contains a hetero-atom group attached by a double bond at the first carbon atom of the side chain that is directly attached to the aromatic ring carbon position, provide skin whitening benefits. The structures of such hydroxyaryl compounds are depicted in FIG. 1. The skin whitening effect is further increased if the hydroxyl group of the hydroxyaryl derivative is on a carbon atom adjacent to the carbon atom that also contains aforementioned carbon side chain with a hetero-atom group attached by a double bond at the first carbon atom of the side chain that is directly attached to the aromatic ring carbon position. The skin whitening effect is frequently enhanced further if additional hydroxy groups are also present on the aromatic ring. Moreover, the skin whitening effect is further enhanced if small amounts of divalent and polyvalent metal ions (such as copper, zinc, manganese, selenium, and vanadium) are also present. Additionally, the hydroxyaryl alkyl ketone derivatives of the present invention provide a surprising and unexpected dual benefit: a skin whitening effect, and an anti-inflammatory effect.

### DETAILED DESCRIPTION

A number of hydroxy acetophenone compositions obtained from natural plant sources have been disclosed in the prior art with antioxidant and other benefits. For example, acetophenone derivatives such as Paeonol (3-hydroxy-5-methoxy acetophenone), 2,5-Dihydroxy-4-Methoxy Acetophenone, and 2,5-Dihydroxy-4-Methyl Acetophenone, have been obtained from Chinese peony. Quinacetophenone (2-acetyl hydroquinone) has been obtained from primrose (Primula Ovalifolia). Scutellarin and Scutellarein (hydroxy benzopyranones) have been obtained from Scutellaria plants. Xanthoxyline (2-hydroxy-4,6-dimethoxyacetophenone) has been isolated from Sebastiania schottiana. Acetophenone derivatives, such as 1-(3-Hydroxy-4-methoxy-5-methylphenyl) ethanone and 1-(3-hydroxy-4-methoxyphenyl) ethanone have been identified from stem bark of Lamprothamnus zanguebaricus. Apocynin (4-hydroxy-3-methoxyacetophenone), is a well-known acetophenone derivative isolated from the traditional medicinal plant Picrorhiza kurroa. 4-Hydroxyacetophenone has been obtained from Ligularia vellerea. These acetophenone derivatives are known for their antioxidant, microcirculation improvement, anti-inflammatory, MAO inhibition, and histamine suppression benefits. Surprisingly and unexpectedly, it has now been found that these acetophenone derivatives provide excellent skin whitening benefits. The skin whitening effect is further increased if certain divalent and polyvalent metal ions, such as copper, zinc, selenium, or vanadium, are also included in combination with such acetophenone derivatives, especially if the hydroxyl group is present on an aromatic ring carbon atom adjacent to the acetyl group. Moreover, conversion of acetyl group of acetophenone and carbonyl ketone group of hydroxyaryl alkyl ketone derivatives mentioned herein into an oxime derivative or a hydrazone derivative or a samicarbazone derivative or an oxamic hydrazone derivative still maintains the skin whitening effect. These chemical structural criteria are further illustrated in FIG. 1.

This is both surprising and unexpected since oxime derivatives of certain hydroxyaryl alkyl ketones have been disclosed in U.S. Patent Application 20030049287 (Ley at al.) as antioxidants, and not as skin whitening agents. The addition of metal ions, such as copper, zinc, or vanadium to such oxime derivatives additionally increases the skin whitening effect of such oxime derivatives.

Also, quite surprisingly, the hydroxyaryl alkyl ketone derivatives of the present invention do not cause any inflammatory skin reactions, like that of hydroquinone. in fact, hydroxyaryl alkyl ketone derivatives of the present invention can actually reduce the skin irritation caused by hydroquinone. This is even further surprising, since many of the hydroxyaryl alkyl ketone derivatives of the present invention have 1,4-dihydroxybenzene chemical structure that is very similar to 1,4-dihydroxybenzene chemical structure of hydroquinone. This unprecedented discovery now permits the combination of hydroquinone itself with hydroxyaryl alkyl ketone derivatives of the present invention. Such combinations synergistically enhance skin whitening effect of hydroquinone. Moreover, the skin irritation that would otherwise be expected from hydroquinone is also reduced significantly. The hydroxyaryl alkyl ketone derivatives of the present invention thus provide a surprising and unexpected dual benefit; a skin whitening effect, and an anti-inflammatory effect. This dual benefit is synergistically extended to other well known skin whitening agents also, such as Arbutin, Kojic acid, and Phytic acid, when such compositions are used in combination with hydroxyaryl alkyl ketone derivatives of the present invention.

The skin whitening effect is not limited to acetophenone derivatives. I have discovered that hydroxyaryl derivatives that contain a carbon side chain directly attached to the aromatic ring carbon, and this carbon chain also contains a hetero-atom group attached by a double bond at the first carbon atom of the side chain that is directly attached to the aromatic ring carbon position, provide a surprising and unexpected complementary and synergistic skin whitening effect. Thus, flavones, chromanones, isoflavones, coumarins, uronates, chromones, and like all provide such complementary and synergistic skin whitening effect. This is quite surprising, since most of such derivatives are known to elicit anti-inflammatory benefits in prior art. The examples of such derivatives that provide good complementary and synergistic skin whitening effect include Puerarin (an isoflavonold), Naringenin (a flavonold), Osthol (a coumarin), Baicalin (an uronate), Baiclein (an uronate), Scutellarin (a flavonoid), Scutellarein (a flavonold), Galangin (a flavonoid) and Paeonlflorin. in addition to providing aforementioned skin whitening effect (which is not known in the prior art), many of these derivatives also provide anti-inflammatory benefits, which is already known in the prior art. The skin whitening property of compositions of present invention can be further boosted by the inclusion of at least one antioxidant composition that can donate a sulfhydryl (—SH) group. This is both surprising and unexpected, since most tyrosinase inhibitors themselves possess antioxidant properties. The examples of sulfhydryl (—SH) donating compositions are glutathione, N-acetyl-cysteine, cysteine, N-acetyl-cystine, cystine, cysteinyl peptides, cystinyl peptides, N-acetyl-S-carboxymethyl-cysteine, Bis-(N-acetyl-S-carboxymethyl)-cystine, and N-Acetyl-4-S-cysteaminylphenol.

The antioxidants of non-sulfhydryl donating groups can also enhance skin whitening efficacy of compositions of the present invention albeit to a lesser degree. Relative to the nature of such non sulfhydryl antioxidant compositions, the selection can be made from Ascorbic acid, Ascorbic acid Esters, Ascorbic acid glucosides, Ascorbic acid salts and other derivatives, Glucosamine ascorbate, Arginine ascorbate, Lysine ascorbate, Glutathione ascorbate, Nicotinamide ascorbate, Niacin ascorbate, Allantoin ascorbate, Creatine ascorbate, Creatinine ascorbate, Chondroltin ascorbate, Chitosan ascorbate, DNA Ascorbate, Carnosine ascorbate, Vitamin E, various Vitamin E derivatives, Tocotrienol, Rutin, Quercetin, Hesperedin (Citrus sinensis), Diosmin (Citrus sinensis), Mangiferin (Mangifera indica), Mangostin (Garcinia mangostana), Cyanidin (Vaccinium myrtillus), Astaxanthin (Haematococcus algae), Lutein (Tagetes patula), Lycopene (Lycopersicum esculentum), Resveratrol (Polygonum cuspidatum), Tetrahydrocurcumin (Curcuma longa), Rosmarinic acid (Rosmarinus officinalis), Hypericin (Hypericum perforatum), Ellagic acid (Punica granatum), Chlorogenic acid (Vaccinium vulgaris), Oleuropein (Olea europaea), alpha-Lipoic acid, Pycnogenol, Grape Seed Extract, Niacinamide lipoate, Glutathione, Andrographolide (Andrographis paniculata), Carnosine, Niacinamide, Potentilla erecta extract, Polyphenols, Grapeseed extract, Pycnogenol (Pine Bark extract), pyridoxine, Horse Chestnut Extract (Aesculus hippocastanum extract)), Esculin, Escin, Yohimbine, Capsicum Oleoresin, Capsaicin, Niacin, Niacin Esters, Methyl Nicotinate, Benzyl Nicotinate, Ruscogenins (Butchers Broom extract; Ruscus aculeatus extract), Diosgenin (Trigonella foenumgraecum, Fenugreek), Emblica extract (Phyllanthus embilca extract), Asiaticoside (Centelia asiatica extract), Boswellia Extract (Boswellia serrata), Ginger Root Extract (Zingiber Officianalls), Piperine, Vitamin K, Meillot (Melilotus officinalis extract), Glycyrrhetinic acid, Ursolic acid, Sericoside (Terminalia sericea extract), Darutoside (Slegesbeckia orientalis extract), Amni visnaga extract, extract of Red Vine (Vitis Vinifera) leaves, apigenin, phytosan, luteolin, Ecklonia cava extract, Spondias mombin extract, Maprounea gulanensis extract, Walteria indica extract, Gouania blanchetiana extract, Cordia schomburgkil extract, Randia armata extract, Hibiscus furcellatus extract, Kaempferia galanga extract, honokiol, magnolol, aminobenzoic acid, Cinoxate, Ethylhexyl methoxycinnamate, Avobenzone, Homosalate, Lawsone, Menthyl anthranilate, Octocrylene, Ethylhexyl salicylate, oxybenzone, Padimate-O, Benzophenone-3, Benzophenone-4, Sulisobenzone, Trolamine salicylate, Glyceryl aminobenzoate, and combinations thereof.

The exact biochemical mechanism by which hydroxyaryl alkyl ketone derivatives of the present invention provide skin whitening effect is not clear at this time. However, it is my hypothesis that the hydroxyaryl alkyl ketone derivatives of the present invention operate by a combination of several mechanisms that at least Includes;, (1) The competitive replacement of tyrosinase substrates, tyrosine and other biochemically related intermediates of tyrosinase substrates, (2) Changes in the environment of copper-copper linkage at the active-site of tyrosinase that can result in the distortion of 26 Angstrom distance between those two copper atoms (II) coupled state of tyrosinase, (3) The irreversible inactivation, replacement, or change in the metal-to-metal oxidation state of copper and zinc ion active-sites of TRP-1 and TRP-2. These mechanisms lead to the blocking of dark colored Eumelanin, and (4) Inhibition of melanocyte stimulating hormone (MSH). it is important to note that chemical structure with a hydroxyaryl or polyhydroxyaryl chemical backbone that also contains an alkyl carbon side chain with a hetero-atom group attached by a double bond at the first carbon atom of the alkyl side chain that is directly attached to the aromatic ring (FIG. 1) is a key requirement, as such chemical structures can also effectively complex with divalent and polyvalent metal ions (which is the reason for my theory that the compositions of the present invention dislocate the copper-copper and zinc-zinc coordination in the active site of Tyrosinase enzyme to effectively inactivate that enzyme for skin whitening effect), as illustrated in FIG. 2.

[FIG. 2. Metal Chelates of Hydroxyaryl and Polyhydroxyaryl Alkyl Ketones and Derivatives]

The compositions of the present invention can also increase the skin whitening efficacy of prior art compositions, many of which are currently marketed commercial products. This is further illustrated in the Examples section of present invention (Examples 5 to 13). The additional examples of such skin whitening agents are mentioned in U.S. Patent Application 20030207776 (A. Shefer et al.), which include adapalene, aloe extract, alpha-Glyceryl-L-ascorbic acid, aminotyroxine, ammonium lactate, anethole derivatives, apple extract, arbutin, areca catechu L. extract, ascorbic acid, ascorbyl palmitate, azelaic acid, bamboo extract, bearberry extract, bletilla tuber, bupleurum falcatum extract, burnet extract, Burnet Power (available from Barnet Products), butyl hydroxy anisole, butyl hydroxy toluene, butyl resorcinol, Chuanxiong, cola decaballo extract, Dang-Gul, deoxyarbutin, 1,3 diphenyl propane derivatives, 2,5 dihydroxybenzoic acid and its derivatives, 2-(4-acetoxyphenyl)-1,3 dithane, 2-(4-hydroxyphenyl)-1,3 dithane, ellagic acid, escinol, estragole derivatives, esculoside, esculetin, FADEOUT (available from Pentapharm), Fangfeng, fennel extract, gallic acid and its derivatives, ganodenna extract, gaoben, GATULINE WHITENING (available from Gattlefosse), genistic acid and its derivatives, gentisyl alcohol, glabrldin and its derivatives, gluco pyranosyl-1-ascorbate, gluconic acid, glucosamine, glycolic acid, glycyrrhizinic acid, green tea extract, 4-Hydroxy-5-methyl-3[2H]-furanone, hydroquinine, 4 hydroxyanisole and its derivatives, 4-hydroxy benzoic acid derivatives, hydroxycaprylic acid, hyptis extract, inositol ascorbate, kojic acid, kojic dipalmitate, lactic acid, lemon extract, licorice extract, Licorice P-TH (available from Barnet Products), linoleic acid, magnesium ascorbyl phosphate, Melfade (available from Pentapharm), MELAWHITE (available from Pentapharm), Melanostatine DM (available from Laboratories Seporga), morus alba extract, mulberry root extract, niacinamide, 5-octanoyl salicylic acid, parsley extract, phellinus linteus extract, pinon blanco extract, pinon negro extract, piri-pirl extract, pyrogallol derivatives, retinoic acid, retinol, retinyl esters (acetate, proplonate, palmitate, linoleate), 2,4 resorcinol derivatives, 3,5 resorcinol derivatives, rose fruit extract, rucinol, salicylic acid, Song-Yi extract, Sophora Powder (available from Barnet Products), 4-thioresorein, 3,4,5 trihydroxybenzyl derivatives, tranexamic acid, tyrostat (Rumex Extract available from Fytokem), Tyroslat 10,11 (available from Fytokem), vanilla derivatives, vitamin D.sub.3 and its analogs, and mixtures thereof.

For the determination of skin whitening and skin lightening effect, a new methodology has been developed in the present invention. Human volunteers were selected with both light and medium dark skin types. An area on the forearm was marked with 1-inch square boxes. The products under test and controls were applied to these areas on an equal weight basis. These areas were then exposed to artificial solar lamp equipment (solar simulator equipment) that is very similar to that used for the SPF determination of sunscreen products. These areas were exposed to artificial sunlight for a period of time that only produced skin-darkening effect on an untreated area without causing any erythema. The treated areas were then exposed to artificial solar light for the same amount of time. The skin lightening effect was then determined both visually and by a light meter reading. In all cases, the skin areas that showed lesser amount of darkening than the corresponding controls were considered as positive response results. in all cases illustrated in the Examples section, the compositions made according to claims section of the present invention showed lighter colored skin (less darkening) than the corresponding control samples that did not contain such compositions. The skin lightening effect was observed on a scale of 1 to 5 (5 means highest amount of skin lightening effect), the differences were at least one scale unit (i.e. observable with naked eye).

### EXAMPLES

The following examples are presented to illustrate presently preferred practice thereof. These examples also include the formulation of consumer desirable lotion, cream, and other such compositions for their retail marketing. As Illustrations they are not intended to limit the scope of the invention. All quantities are in weight %.

### Example 1

Skin Lightener Serum (According to claim 1). ingredients % Weight (1) Deionized water 20.0 (2) Quinacetophenone 5.0 (3) Methylpropanediol 69.5 (4) Dimethicone copolyol 4.0 (5) Preservatives 0.5 (6) Ammonium Acryloyidimethyltaurate/vp copolymer 1.0. Procedure. Make main batch by mixing (2) to (5) at room temperature. Pre-mix (1) and (6) to a clear paste and add to main batch with mixing. The product has a clear to slightly hazy syrup-like appearance, typical of a skin serum product. it is absorbed rapidly with a silky smooth skin feel. A skin lightening effect is observed by the test method described in Example 14 section of the present invention.

### Example 2

Skin Lightener Serum with Copper ions (According to claim 6). ingredients % Weight (1) Delonized water 20.0 (2) Quinacetophenone 5.0 (3) Methylpropanedlol 69.0 (4) Dimethicone copolyol 4.0 (5) Preservatives 0.5 (6) Copper Gluconate 0.5. (7) Ammonium Acryloyidimethyltaurate/vp copolymer 1.0 Procedure. Make main batch by mixing (2) to (6) at room temperature. Pre-mix (1) and (7) to a clear paste and add to main batch with mixing. The product has a clear to slightly hazy syrup-like light blue appearance, typical of a skin serum product. it is absorbed rapidly with a silky smooth skin feel. A skin lightening effect is observed by the test method described in Example 14 section of the present invention.

### Example 3

Skin Lightener Cream (According to claim 2). ingredients % Weight (1) Deionized water 79.5 (2) Cetearyl alcohol (and) dicetyl phosphate (and) Ceteth-10 phosphate 5.0 (3) Cetyl alcohol 2.0 (4) Glyceryl stearate (and) PEG-100 stearate 4.0 (5) Caprylic/capric triglyceride 5.0 (6) Resacetopheenone 3.0 (7) Paeonol 1.0 (8) (8) Preservatives 0.5. Procedure. Mix 1 to 5 and heat to 75-80° C. Adjust pH to 4.0 4.5. Cool to 35-40° C. with mixing. Add 6 to 8 with mixing. Adjust pH to 4.0-4.5, if necessary. White to off-white cream. A skin lightening effect is observed by the test method described in Example 14 section of the present invention.

### Example 4

Skin Depigmentation Facial Mask Composition ingredient (according to claim 10). % (1) Chitosan 5.0 (2) 2,5-Dihydroxy acetophenone Oxime 5.0 (3) Glycerin 17.7 (4) Water 70.6 (5) Yohimbine HCl 0.5 (6) Niacinamide Lipoate 0.5 (7) Glutathione0.2 (8) Preservatives 0.5 Procedure: Mix 1, 2, and 3 to a paste. Mix 4 to 8 separately to a clear solution. Add this to main batch and mix. A clear gel product is obtained. it is applied on the face and neck and left for 10 to 30 minutes, then rinsed off. A skin lightening effect is observed by the test method described in Example 14 section of the present invention.

### Example 5

Boosting Skin Whitening activity of “Gigawhite” Composition with a Composition from claim 1. Ingredient % (1) “Gigawhite” (obtained from Alpaflor Company, Switzerland) 90.0 (2) PEG-6 9.5 (3) Resacetophenone 0.5 Procedure. Mix 2 and 3 to a solution. Add to 1 and mix. A thin, light brown liquid is obtained. A skin lightening effect is observed by the test method described in Example 14 section of the present invention.

### Example 6

Boosting Tyrosinase inhibiting property of “Tyrostat-09” Composition with a Composition from claim 1. Tyrostat-09 composition was obtained from Fytokem, Saskatchewan, Canada. ingredient % (1) Paeonol 5.0 (2) Propylene Glycol 10.0 (3) Tyrostat-09 85.0. Procedure. Mix (1) and (2) to a solution. Add this solution to (3) and mix. A light amber liquid is obtained. A skin lightening effect is observed by the test method described in Example 14 section of the present invention.

### Example 7

Boosting Tyrosinase inhibition of “Etioline” composition with a Composition from claim 1.“Etioline” was obtained from Sederma/Croda, USA. ingredient % (1) 2,6-Dihydroxy Acetophenone 5.0 (2) Propylene Glycol 10.0 (3) Etioline 85.0 Procedure. Mix (1) and (2) to a solution. Add to (3) and mix. An amber solution was obtained. A skin lightening effect is observed by the test method described in Example 14 section of the present invention.

### Example 8

Boosting Anti-Tyrosinase action of “Gatuline Whitening” composition with a Composition from claim 1. “Gatulin Whitening” was obtained from Gattefosse Corporation. ingredient % (1) 2,5-Dihydroxy acetophenone (Quinacetophenone) 2.5 (2) Ethanol 7.5 (3) Gatulin Whitening 90.0 Procedure. Mix (1) and (2) to a solution. Add this to (3) and mix. A light amber solution was obtained. A skin lightening effect is observed by the test method described in Example 14 section of the present invention.

### Example 9

Boosting Tyrosinase inhibitory activity of “Dermalight” Composition with a Composition from claim 1. “Dermalight” was obtained from Silab, France. ingredient % (1) 2,5-Dihydroxy acetophenone copper complex 1.5 (2) Deionized water 5.0 (3) “Dermalight” 93.5. Procedure. Mix (1) and (2) to a solution. Add this to (3) and mix. An amber solution was obtained. A skin lightening effect is observed by the test method described in Example 14 section of the present invention.

### Example 10

Boosting Anti-tyrosinase activity of “Glgawhite” Composition according to claim 11. ingredient % (1) “Gigawhite” (obtained from Alpaflor Company, Switzerland) 89.5 (2) PEG-6 9.5 (3) 2,6-Dihydroxy Propiophenone 0.5 (4) Glutathione 0.5. Procedure. Mix (2), (3) and (4) to a solution. Add to (1) and mix. A thin, light brown liquid is obtained. A skin lightening effect is observed by the test method described in Example 14 section of the present invention.

### Example 11

Boosting Tyrosinase inhibiting property of “Tyrostat-09” Composition according to Combination of claim 1, claim 10, and claim 15. Tyrostat-09 composition was obtained from Fytokem, Saskatchewan, Canada. Ingredient % (1) 2,5-Dihydroxy acetophenone 5.0 (2) 2,5-Dihydroxy Acetophenone Oxime 0.5 (3) S-Carboxymethyl-N-Acetyl-Cystelne 0.5 (4) Propylene Glycol 9.5 (5) Tyrostat-09 84.5. Procedure. Mix (1), (2), (3), and (4) to a solution. Add this solution to (5) and mix. A light amber liquid is obtained. A skin lightening effect is observed by the test method described in Example 14 section of the present invention.

### Example 12

Boosting Tyrosinase inhibition of “Etioline” Composition according to Combination of claim 10 and claim 7. “Etioline” was obtained from Sederma/Croda, USA. Ingredient % (1) Resacetophenone oxime 5.0 (2) Glutathione 0.5 (3) Deionized water 10.0 (4) Etioline 84.5 Procedure. Mix (1), (2) and (3) to a solution. Add to (4) and mix. An amber solution was obtained. A skin lightening effect is observed by the test method described in Example 14 section of the present invention.

### Example 13

Boosting Anti-Tyrosinase action of “Gatuline Whitening” according to claim 17. “Gatulin Whitening” was obtained from Gattefosse Corporation. Ingredient % (1) Quinacetophenone Oxime 2.5 (2) Benzophenone-4 0.5 (3) Deionized water 7.5 (4) Gatulin Whitening 89.5. Procedure. Mix (1), (2) and (3) to a solution. Add this to (4) and mix. A light amber solution was obtained. A skin lightening effect is observed by the test method described in Example 14 section of the present invention.

### Example 14

Determination of skin whitening and skin lightening effect. A new methodology has been developed in the present invention. Human volunteers were selected with both light and medium dark skin types. An area on the forearm was marked with 1-inch square boxes. The products under test and controls were applied to these areas on an equal weight basis. These areas were then exposed to artificial solar lamp equipment (solar simulator equipment) that is very similar to that used for the SPF determination of sunscreen products. These areas were exposed to artificial sunlight for a period of time that only produced skin-darkening effect on an untreated area without causing any erythema. The treated areas were then exposed to artificial solar light for the same amount of time. The skin lightening effect was then determined both visually and by a light meter reading. in all cases, the skin areas that showed lesser amount of darkening than the corresponding controls were considered as positive response results. in all cases illustrated in the Examples section, the compositions made according to claims section of the present invention showed lighter colored skin (less darkening) than the corresponding control samples that did not contain such compositions. The skin lightening effect was observed on a scale of 1 to 5 (5 means highest amount of skin lightening effect), the differences were at least one scale unit (i.e. observable with naked eye).

1. A topical skin depigmenting or skin whitening composition comprising;
(i) At least one hydroxyaryl or polyhydroxyaryl composition that contains an alkyl carbon side chain with a hetero-atom group attached by a double bond at the first carbon atom of the alkyl side chain that is directly attached to the aromatic ring, and
(ii) A cosmetically or pharmaceutically acceptable topical delivery system or carrier base composition.

2. A composition according to claim 1, wherein hydroxyaryl or polyhydroxyaryl composition is selected from hydroxy or polyhydroxy acetophenones.

3. A composition according to claim 1, wherein hydroxyaryl or polyhydroxyaryl composition is selected from hydroxy or polyhydroxy Aryl Alkyl Ketones.

4. A composition according to claim 1, wherein cosmetically or pharmaceutically acceptable topical delivery system or carrier base composition additionally contains hydroxy or polyhydroxy flavones, hydroxy or polyhydroxy coumarins, hydroxy or polyhydroxy isoflavones, hydroxy or polyhydroxy chromanones, and hydroxy or polyhydroxy chromones, and combinations thereof.

5. A composition according to claim 1, wherein cosmetically or pharmaceutically acceptable topical delivery system or carrier base composition additionally contains hydroquinone, arbutin, Kojic acid, Phytic acid, and combinations thereof.

6. A composition according to claim 1, wherein a cosmetically or pharmaceutically acceptable topical delivery system or carrier base composition additionally contains a divalent or a polyvalent metal ion or combinations thereof.

7. A composition according to claim 1, wherein a cosmetically or pharmaceutically acceptable topical delivery system or carrier base composition additionally contains a sulfhydryl or thiol (—SH) group donating antioxidant composition.

8. The compositions according to claim 1, wherein the cosmetically or pharmaceutically acceptable delivery system can be traditional water and oil emulsions, suspensions, colloids, microemulsions, clear solutions, suspensions of nanoparticles, emulsions of nanoparticles, powders, or anhydrous compositions.

9. A composition according to claim 2, wherein hydroxy or polyhydroxy acetophenone composition is selected from 2-hydroxyacetophenone, 3-hydroxyacetophenone, 4-hydroxyacetophenone, 2,3-dihydroxyacetophenone, 2 ,4-dihydroxyacetophenone, 2,5-dihydroxyacetophenone, 2,6-dihydroxyacetophenone, 3,4-dihydroxyacetophenone, 3,5-dihydroxyacetophenone, 2,4,6-trihydroxyacetophenone, 2,3,4-trihydroxyacetophenone, 2,3,5-trihydroxyacetophenone, 2,3,6-trihydroxyacetophenone, 2,4,5-trihydroxyacetophenone, 3,4,5-trihydroxyacetophenone, Resacetophenone, 2-Acetyl resorcinol, 4-Acetyl resorcinol, 3,4-Dihydroxyacetophenone, acetyl quinol, Quinacetophenone, 1-(3-Hydroxy-4-methoxy-5-methylphenyl) ethanone, 1-(3-hydroxy-4-methoxyphenyl) ethanone, Paeonol, 5′-Bromo-2′-hydroxyacetophenone, 5′-Chloro-2′-hydroxyacetophenone, 3′,5′-Dlchloro-2′-hydroxyacetophenone, 3′,5′-Dibromo-4′-hydroxyacetophenone, 5-Chloro-3-bromo-2-hydroxyacetophenone, and combinations thereof.

10. A composition according to claim 2, wherein oxime, or oxime O-alkyl ether, or hydrazone, or semicarbazone, or oxamic hydrazone derivatives of hydroxy or polyhydroxy acetophenones, or hydroxy or polyhydroxy aryl alkyl ketones or combinations thereof are selected.

11. A composition according to claim 3, wherein hydroxy or polyhydroxy Aryl Alkyl Ketones composition is selected from 2-hydroxypropiophenone, 3-hydroxypropiophenone, 4-hydroxyproplophenone, 2,3-dihydroxypropiophenone, 2,4-dihydroxypropiophenone, 2,5-dihydroxyproplophenone, 2,6-dihydroxyproplophenone, 3,4-dihydroxyproplophenone, 3,5-dihydroxyproplophenone, 2,4,6-trihydroxypropiophenone, 2,3,4-trihydroxyproplophenone, 2,3,5-trihydroxypropiophenone, 2,3,6-trihydroxypropiophenone, 2,4,5-trihydroxypropiophenone, 3,4,5-trihydroxyproplophenone, and combinations thereof.

12. A composition according to claim 3, wherein oxime, or oxime O-alkyl ether, or hydrazone, or semicarbazone, or oxamic hydrazone derivatives of hydroxy or polyhydroxy propiophenones, and combinations thereof are selected.

13. A composition according to claim 4, wherein hydroxyaryl or polyhydroxyaryl composition is further selected from Xanthoxyline, Apocynin, Hydroxyacetophenone, Scutellarin, Scutellarein, Baicalin, Galangin, Balcalein, Puerarin, Puerarein, Naringenin, Osthol, Paeonoflorin, and combinations thereof.

14. A composition according to claim 6, wherein divalent metal ions are selected from copper, zinc, iron, selenium, vanadium, manganese and combinations thereof.

15. A composition according to claim 7, wherein one sulfhydryl (—SH) donating composition is selected from glutathione, N-acetyl-cysteine, cystelne, N-acetyl-cystine, cystine, cysteinyl peptides, cystinyl peptides, N-Acetyl-4-S-cysteaminylphenol, and combinations thereof.

16. A composition according to claim 8, wherein cosmetically or pharmaceutically acceptable delivery system or carrier base can optionally include additional skin beneficial ingredients selected from skin cleansers, surfactants (catlonic, anionic, non-ionic, amphoteric, and zwitterionic), skin and hair conditioning agents, vitamins, hormones, minerals, plant extracts, anti-inflammatory agents, concentrates of plant extracts, emollients, moisturizers, skin protectants, humectants, silicones, skin soothing ingredients, analgesics, skin penetration enhancers, solubilizers, moisturizers, emollients, anesthetics, colorants, perfumes, preservatives, seeds, broken seed nut shells, silica, clays, beads, luffa particles, polyethylene balls, mica, pH adjusters, processing aids, and combinations thereof.

17. A composition according to claim 10, wherein oxime derivatives of hydroxyaryl compositions are selected from 2-hydroxyacetophenone oxime, 2,3dihydroxyacetophenone oxime, 2,4-dihydroxyacetophenone oxime, 2,5-dihydroxyacetophenone oxime, Resacetophenone oxime, acetyl quinol oxime, Quinacetophenone oxime, Paeonol oxime, 2-hydroxyproplophenone oxlme, 2,3-dihydroxypropiophenone oxime, 2,4-dihydroxypropiophenone oxime, 2,5-dihydroxypropiophenone oxime, 7-acetyl-5,8-dihydroxyquinoline oxime, and combinations thereof.

18. A composition according to claim 16, wherein anti-inflammatory compositions are selected from Boswellia serrata, Corosolic acid (Banaba), Ursolic acid, Oleanolic acid, Salicinol (Salacia), Rosmarinic acid, Ruscogenins, Darutoside, Aslaticoside, Sericoside, Harpagoside (Devil's Claw), Magnolia Bark (Honokiol, Magnolol), Horse Chestnut (Escin, Esculin), Ginger (Gingerol), Turmeric Extract (Tetrahydrocurcuminolds), Corydalls, Myricetin, and combinations thereof.
